(19) United States
(12) Patent Application Publication
(10) Pub. No.: US 2023/0076310 A1
(43) Pub. Date: Mar. 9, 2023

(54) COMPOSITION USING NOVEL LACTOBACILLUS PLANTARUM KC3 STRAIN FOR PREVENTION OR TREATMENT OF IMMUNE IMPAIRMENT, RESPIRATORY INFLAMMATORY DISEASE, ALLERGY, AND ASTHMA AND USE THEREOF

(71) Applicants: KT&G CORPORATION, Daejeon (KR); CKD BIO CORPORATION, Seoul (KR)

(72) Inventors: Han Jae SHIN, Daejeon (KR); Moon Yong LEE, Daejeon (KR); Young Sin KIM, Yongin-si (KR); Do Hoon KIM, Daejeon (KR); Dong Hoon LEE, Daejeon (KR); In Ock LEE, Incheon (KR); Min Ho SEO, Gwangmyeong-si (KR); Bo Hye HEO, Ansan-si (KR); Woo Ri KIM, Incheon (KR); Byoung Kook KIM, Seoul (KR); In Suk CHOI, Seoul (KR)

(73) Assignees: KT&G CORPORATION, Daejeon (KR); CKD BIO CORPORATION, Seoul (KR)

(21) Appl. No.: 17/797,891

(22) PCT Filed: Jan. 26, 2021
(86) PCT No.: PCT/KR2021/001024

§ 371 Date: Aug. 5, 2022

(30) Foreign Application Priority Data
Feb. 13, 2020 (KR) 10-2020-0017691

(51) Int. Cl. A61K 35/747; A61P 11/00; A23L 33/135; A23L 2/52

(52) U.S. Cl. CPC A61K 35/747; A61P 11/00; A23L 33/135; A23L 2/52; A23V 2002/00; A23Y 2220/67

(57) ABSTRACT

The present disclosure relates to a composition for ameliorating immune disorders and preventing or treating respiratory inflammatory diseases, the composition including, as an active ingredient, new lactic acid bacteria from Lactobacillus plantarum KC3 (also referred to as “CKDB-KC3”). It is confirmed that the composition is useful as a pharmaceutical composition or a health functional food for ameliorating immune disorders and preventing and treating a respiratory inflammatory diseases by confirming that the new strain of the present disclosure exhibits excellent activities of ameliorating immune disorders and inhibiting respiratory inflammatory diseases through the following animal experiments: an experiment on the characteristics of the new lactic acid bacteria from the Lactobacillus plantarum KC3 (see Experimental Example 1); an experiment on the inhibitory effect of probiotics on the expression of inflammatory cytokines in the intestines (see Experimental Example 2); an experiment on the anti-inflammatory effect on ear edema (see Experimental Example 3); and an experiment on the defense effect against respiratory damage caused by air pollutants such as fine dust (see Experimental Example 4 and FIG. 1). That is, the new strain disclosed herein may be usefully utilized as a pharmaceutical composition, health functional food, or health supplement food for the amelioration of immune disorders and the prevention or treatment of respiratory inflammatory diseases.

### TECHNICAL FIELD

The present disclosure relates to a composition for preventing or treating immune disorders and respiratory inflammatory diseases and use of the composition, the composition including a new Lactobacillus plantarum KC3 strain as an active ingredient.

### BACKGROUND ART

The human body has characteristics of maintaining homeostasis, and such characteristics also apply to an immune system. The term “immunity” refers to a series of biodefence reactions that occur in a living body to exclude substances, other than its own substances, which disrupt the homeostasis of the living body or are a threat to the body. Immunity also refers to a defense system that protects the body from harmful substances that have invaded through the skin, digestive tract, respiratory tract, and the like, and may be said to be an activity maintaining homeostasis through phagocytosis which removes external irritants or the reduction of severe inflammation of injuries. All immune responses including inflammatory reactions that have activated by the inflow of pathogens into the body return to a normal state when the pathogens are removed. However, in the presence of difficulties with immune regulation, inflammatory reactions occur significantly higher or lower than those of people having normal immune reactions. A state in which immune functions are deficient or degraded is called “immunodeficiency”, and in this state, immune responses are not activated properly, such that the body fails to properly respond to foreign substances, leading to infection. On the other hand, a “hypersensitivity reaction” refers to an overreaction of some immune responses, resulting in an imbalance in the immune system, thereby causing an allergic reaction (Immunology. 49, 1992; Dig dis Sci. 52, pp 1890-1896, 2007; J Life Sci. 19, pp 479-485, 2009; Biol Pharm Bull. 27, pp 617-620, 2004).

When the immune system is not regulated normally and becomes imbalanced, disorders of immune regulation occur due to imbalanced cytokines in the body, imbalanced proliferation of T/B cells, depletion of antioxidant nutrients, and the like. Accordingly, the secretion of inflammatory substances increases, causing damage to cells and tissues, and due to a failure to cope with the inflow of pathogens, the inflammation may become more severe. Therefore, the immune responses of the human body should be balanced and regulated normally to maintain health, and in this regard, the ability to regulate immunity is considered important for prophylaxis and treatment of diseases (J Allergy clin immunol. 9, pp 616, 1993; Immunology. 47, pp 75, 1982).

The spleen is involved in immunoregulation, and cytokines, such as interferon (IFN)-γ, tumor necrosis factor (TNF)-α, and interleukin (IL)-1β, IL-5, IL-6, PGE2, and the like, are secreted in the spleen. Such cytokines show activity in the execution stage of an immune response, and play an important role in a signal transduction between immune cells and the inflammatory system.

Since 2000, new acute infectious diseases, such as severe acute respiratory syndrome (SARS), avian influenza, and Ebola virus, have emerged around the world, and the importance of immunity, which is the ability of the body to defend against such infectious diseases, is further emerging. Immunity, which is a biological defense against threats including breaking of the homeostasis of the human body or the invasion of pathogens, may be classified into non-specific immunity associated with macrophages or natural killer (NK) cells and specific immunity associated with T or B cells (1. A-Reum Yu, Ho-Young Park, Yun-Sook Kim, Sang-Keun Ha, Hee-Do Hong, Hee-Don Choi. 2012. Immuno-enhancing Effect of Seed Extracts on a RAW 264.7 Macrophage Cell Line. J Korean Soc Food Sci Nutr 41(12): 1671-1676). When pathogens invade the human body, phagocytes such as neutrophils, monocytes, and macrophages are major cell groups for innate immune responses, and first defend the human body against the pathogens (Uthaisangsook S, Day N K, Bahna S L, Good R A, Haraguchi S. 2002. Innate immunity and its role against infections. Ann. Allergy Asthma Immunol. 88: 253-264). In particular, the macrophages are the first reactive cells in the biodefense after the epithelial cell barriers. The macrophages also perform functions as antigen presenting cells and influence the action of T cells in relation to adaptive immunity (3. Birk R W, Gratchev A, Hakiy N, Politz O, Schledzewski K, Guillot P, Orfanos C E, Goerdt S. 2001. Alternative activation of antigen-presenting cells: concepts and clinical relevance. Hautarzt 52: 193-200). The macrophages can also remove pathogen-infected cells or cancer cells, and secrete cytokines, such as nitric oxide (NO) and tumor necrosis factor-α (TNF-α) that contribute to immune responses (4. Seon A Yoo, Ok Kyung Kim, Da-Eun Nam, Yongjae Kim, Humyoung Baek, Woojin Jun, Jeongmin Lee. 2014. Immunomodulatory Effects of Fermented Curcuma longa L. Extracts on RAW 264.7 Cells. J Korean Soc Food Sci Nutr 43(2): 216-243).

Generally, an inflammatory response is a defensive response process of a living body associated with restoration and regeneration of a damaged part, when an invasion causing some organic changes in the cells or tissues of the living body is applied. Thus, spots for a series of such reactions include local blood vessels, various tissue cells in body fluids, immune-related cells, and the like. With recent developments in molecular biology, attempts have been made to understand the involvement of cytokines in inflammatory diseases at a molecular level, and factors affecting such diseases have been investigated one by one.

Allergic reactions may be classified into four categories, i.e., Type I, Type II, Type III, and Type IV, according to types of the allergic reaction. Alternatively, allergies of Type I, Type II, and Type III may be referred to as immediate-type allergies, and allergies of Type IV may be referred to as delayed-type allergies according to a period of time to onset from re-sensitization caused by allergens.

Among the allergies, Type I allergy is a reaction in which IgE antibodies are involved, and is referred to as anaphylaxis-type allergy, and examples of Type I allergy include bronchial asthma, atopic diseases (e.g., dermatitis, gastroenteritis, etc.), allergic rhinitis such as pollinosis, allergic conjunctivitis, food allergies, and the like.

Asthma is a disease characterized by airway hyperresponsiveness to various stimuli, and clinical symptoms including wheezing, dyspnea, and coughing that are caused by airway stenosis may be naturally or reversibly alleviated by treatment. In most cases, asthma is allergic, and is characterized by chronic airway inflammation and bronchial hyperresponsiveness (Minoguchi K and Adachi M. Pathophysiology of asthma. In: Cherniack N S, Altose M D, Homma I, editors. Rehabilitation of the patient with respiratory disease. New York: McGraw-Hill, 1999, pp 97-104).

Asthma has been regarded as a chronic inflammatory disease because inflammatory cells are proliferated, differentiated, and activated by interleukins-4, 5, and 13, which are generated by T-helper2 (TH2) immune cells, and then move and infiltrate into the airway and neighboring tissues thereof (Elias J A, et al., J. Clin. Invest., 111, pp 291-297, 2003). The activated inflammatory cells, such as eosinophils, mast cells, and alveolar macrophages, in the bronchus of patients suffering from asthma, secrete a variety of inflammatory mediators (cysteine leukotriene, prostaglandin, etc.) and are involved in potent bronchial constriction (Maggi E., Immunotechnology, 3, pp 233-244, 1998; Pawankar R., Curr. Opin. Allergy Clin. Immunol., 1, pp 3-6, 2001; Barnes P J, et al., Pharmacol Rev., 50, pp 515-596, 1998).

Accordingly, due to the production and actions of cytokines, such as IL-4, IL-5, and IL-13, and immunoglobulin E, which are involved in the activation of inflammatory cells, biosynthesis of cysteine leukotrienes including eosinophils secreted from inflammatory cells may be the main causes of inflammation, allergic reactions, and asthma caused by inflammation and allergic reactions. Thus, a number of studies have been conducted to develop drugs to inhibit the production of cytokines and immunoglobulin E.

In addition, chronic obstructive pulmonary disease (COPD) should be treated appropriately and distinctly from asthma, which is characterized mainly by reversible airflow obstruction and allergic bronchial inflammatory responses. However, current COPD treatments only provide symptomatic alleviation, and none of the recent treatments have demonstrated fundamental therapeutic effects on COPD as a clinical result (Hele D J, Belvisi M G. 2003. Novel therapies for the treatment of inflammatory airway diseases. Expert Opin Investig Drugs 12: 5-18; Fox J C, Fitzgerald M F. 2009. The role of animal models in the pharmacological evaluation of emerging anti-inflammatory agents for the treatment of COPD. Curr Opin Pharmacol. 9: 231-242).

Asthma and COPD show different pathological mechanisms in principle, and are known to have distinct pathological mechanisms. For example, (1) in terms of inflammatory cells, asthma is mainly associated with mast cells, eosinophils, CD4+ cells (Th2), macrophages, and the like, whereas COPD is mainly associated with neutrophils, CD8+ cells (Tc), and the like; (2) in terms of inflammatory mediators, asthma mainly involves leukotriene B, histamine, IL-4, IL-5, IL-13, eotaxin, RANTES, oxidative stress, and the like, whereas COPD mainly involves TNF-alpha, IL-8, GROalpha, and the like; and (3) in terms of inflammatory phenomenon, asthma acts on the entire airways with airway hyperresponsiveness (AHR), epithelial shedding, fibrosis, no parenchymal involvement, mucus secretion, relative reversible airflow obstruction, coughing, sneezing, and dyspnea usually occurring in childhood, whereas COPD acts on the peripheral airways with epithelial metaplasia, parenchymal destruction, relative irreversible airflow obstruction, chronic bronchitis, and pulmonary emphysema mainly occurring in adulthood (Barnes P J. 2000. Mechanisms in COPD: differences from asthma. Chest 117: 10S-14S; Seatta M, Turato G, Maestrelli P, Mapp C E, Fabbri L M. 2001. Cellular and structural base of chronic obstructive pulmonary disease. Am J Respir Crit Care Med. 163: 1304-1309).

The inventors of the present disclosure have been focusing on the development of therapeutic agents using various resources, particularly natural resources including lactic acid strains of which the safety and efficacy are already well known, as therapeutic agents using antibodies against various types of cytokines and chemokines that are characterized in respiratory inflammatory diseases.

In this regard, the following disclosures have been disclosed: KR 10-2011883 disclosing “Lactobacillus plantarum KC3 strain having anti-obesity effect and uses thereof”; KR 10-2018-0044245 disclosing “Novel lactic acid bacteria having constipation improvement effect and use thereof”; KR 10-2019-0022168 disclosing “Lactobacillus rhamnosus CKDB009 having anti-obesity and cholesterol lowering effect and uses thereof”; KR 10-1794567 disclosing “The composition comprising the specific extract or the purified fraction isolated from Salvia plebeia R. Br. as an active ingredient for preventing or treating respiratory inflammatory disease”; KR 10-1770766 disclosing “The composition comprising the specific extract isolated from Leonurus sibiricus as an active ingredient for preventing or treating respiratory inflammatory disease”; and KR 10-1940042 disclosing “A composition comprising an extract of Salvia plebeia, and red ginseng, as an active ingredient for preventing or treating respiratory inflammatory disease”.

However, none of the documents above discloses or teaches anything about a therapeutic agent for ameliorating immune disorders and respiratory inflammatory diseases, including, as an active ingredient, a new Lactobacillus plantarum KC3 strain isolated from Korean kimchi.

In this regard, the inventors of the present disclosure completed the present disclosure by confirming that the Lactobacillus plantarum KC3 strain exhibits excellent activities of ameliorating immune disorders and inhibiting respiratory inflammatory diseases through the following animal experiments targeting the Lactobacillus plantarum KC3 strain having an anti-obesity effect as disclosed in the existing KR 10-2011883: an experiment on characteristics of new lactic acid bacteria from the Lactobacillus plantarum KC3 (see Experimental Example 1); an experiment on an inhibitory effect of probiotics on expression of inflammatory cytokines in the intestines (see Experimental Example 2); an experiment on an anti-inflammatory effect on ear edema (see Experimental Example 3); and an experiment on a defense effect against respiratory damage caused by air pollutants such as fine dust (see Experimental Example 4).

### DESCRIPTION OF EMBODIMENTS

### Technical Problem

The inventors of the present disclosure aim to develop a more excellent therapeutic agent for ameliorating immune disorders and preventing or treating a respiratory inflammatory disease.

An objective of the present disclosure is to provide a pharmaceutical composition for ameliorating immune disorders and preventing or treating respiratory inflammatory diseases, the pharmaceutical composition including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

Another objective of the present disclosure is to provide a method of ameliorating immune disorders and treating respiratory inflammatory diseases, the method including administering one or more selected from the group consisting of a Lactobacillus plantarum KC3 strain (also referred to as “CKDB-KC3”, Accession number: KCTC13375BP), a culture thereof, a concentrate of the culture, and a dried material of the culture, to a patient having immune disorders and respiratory inflammatory diseases.

Another objective of the present disclosure is to provide use of one or more selected from the group consisting of a Lactobacillus plantarum KC3 strain (also referred to as “CKDB-KC3”, Accession number: KCTC13375BP), a culture thereof, a concentrate of the culture, and a dried material of the culture, for preparation of a therapeutic agent for the amelioration of immune disorders and the treatment of a patient having respiratory inflammatory diseases.

In addition, another objective of the present disclosure is to provide a health functional food for ameliorating immune disorders and preventing or ameliorating respiratory inflammatory diseases, the health functional food including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

In addition, another objective of the present disclosure is to provide a health supplement food for ameliorating immune disorders and preventing or ameliorating respiratory inflammatory diseases, the health supplement food including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

In addition, another objective of the present disclosure is to provide a probiotic agent for ameliorating an immune disorder and preventing or treating a respiratory inflammatory disease, the probiotic agent including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

### Solution to Problem

One of the objectives of the present disclosure is to provide a pharmaceutical composition for ameliorating an immune disorder and preventing or treating a respiratory inflammatory disease, the pharmaceutical composition including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

In addition, as another aspect, the present disclosure provides a health functional food for ameliorating immune disorders and preventing or ameliorating respiratory inflammatory diseases, the health functional food including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

In addition, as another aspect, the present disclosure provides a health supplement food for ameliorating immune disorders and preventing or ameliorating respiratory inflammatory diseases, the health supplement food including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

In addition, another aspect of the present disclosure provides a probiotic agent for ameliorating an immune disorder and preventing or treating a respiratory inflammatory disease, the probiotic agent including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

As another aspect to achieve the objectives above, the present disclosure provides a pharmaceutical composition for ameliorating an immune disorder and preventing or treating a respiratory inflammatory disease, the pharmaceutical composition including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

The Lactobacillus plantarum KC3 strain (also referred to as “CKDB-KC3”) as defined herein does not produce biogenic amines from one or more amino acid precursors selected from the group consisting of tyrosine, histidine, ornithine and lysine.

The present invention provides, as a lactic acid bacterium having efficacy of ameliorating an immune disorder and treating a respiratory inflammatory disease, a Lactobacillus plantarum KC3 strain (also referred to as “CKDB-KC3”, Accession No: KCTC13375BP) including a nucleic sequence of SEQ ID NO: 1 as 16s rRNA.

In an embodiment of the present disclosure provides a Lactobacillus plantarum KC3 strain, which is a new lactic acid bacterium isolated from kimchi, having anti-inflammation efficacy, especially on the amelioration of an immune disorder and the treatment of a respiratory inflammatory disease.

The term “immune disorder” as defined herein includes: a disease caused by a decrease in immune functions due to anticancer therapy such as chemotherapy and radiotherapy or by a decrease in immunity after bone marrow transplantation; AIDS due to damage to an immune system; and a cancer disease due to degradation in immune functions. Preferably, the immune disorder includes a disease caused by a decrease in immune functions due to anticancer therapy such as chemotherapy and radiotherapy or by a decrease in immunity after bone marrow transplantation. More preferably, the immune disorder includes bacterial/viral infection in the elderly, chronic respiratory infection, chronic urinary tract infection, bedsore, flu, pneumonia, chickenpox in children, measles, exanthema subitum, hand-foot-and-mouth disease, rubella, or Crohn's disease.

The term “respiratory inflammatory disease” as defined herein may be any one respiratory inflammatory disease selected from the group consisting of rhinitis, otitis media, sore throat, tonsilitis, pneumonia, asthma, and chronic obstructive pulmonary disease. In detail, the respiratory inflammatory disease may include any one respiratory inflammatory disease selected from the group consisting of rhinitis, otitis media, sore throat, tonsilitis, pneumonia, asthma, and chronic obstructive pulmonary disease that are caused by air pollutants or fine dust, but is not limited thereto.

The terms “a culture of the strain, a concentrate of the culture, and a dried material of the culture” as defined herein are commonly used in the art. Although not limited thereto, the terms refer to various strain cultures including, as an active ingredient, the Lactobacillus plantarum KC3 strain of the present disclosure, a concentrate of the strain culture in which the strain culture is concentrated, and a dried material of the strain culture.

A lysate may refer to a product obtained by fragmentizing the strain itself by applying a chemical or physical force thereto.

The culture may refer to, regardless of the form of a culture, a material including some of or all of materials contained in a medium where the strain is cultured. For example, the culture may refer to a material including a metabolite or a secretion resulting from culturing the strain, or a lysate of the material, and the strain itself may also be contained in the culture. In addition, the culture may refer to inclusion of a fermented product.

An extract may refer to a product obtained by, regardless of an extraction method, an extraction solvent, an extracted component, or an extract type, extracting a strain itself, a lysate of the strain, a culture of the strain, or a mixture thereof. The extract is also a broad concept that includes all materials that can be obtained by processing or treating the extract by using different methods such as fractionation, concentration, and the like after extraction.

Hereinafter, the present disclosure will be described in more detail.

The strain of the present disclosure may be obtained according to the following preparation method.

For example, although not limited thereto, processes of isolating and identifying a microorganism for the Lactobacillus plantarum CKDB-CK3 strain of the present invention may be performed as described below.

The new Lactobacillus plantarum KC3 strain of the present disclosure (also referred to as “CKDB-KC3”) can be obtained from kimchi of different types from different regions in South Korea. Preferably, the new strain can be obtained from kimchi from commercially available products such as Jonggajip™ and CJ Bibigo™, or from home-made Korean kimchi prepared in restaurants, homes, and temples in North Gyeongsang Province, North Chungcheong Province, Gyeonggi Province, North Jeolla Province, South Jeolla Province, and the like. More preferably, the new strain can be obtained from kimchi from Jeonju city in North Jeolla Province. More preferably, although not limited thereto, the new strain can be obtained from kimchi prepared by the following six steps:

Step 1: raw materials for kimchi seasoning are prepared by six stages (a) to (f): (a) (first stage) a Chinese cabbage from North Jeolla Province is prepared and cut into two pieces after getting rid of inedible portions, and then, in a container for salting, salt in an amount in a range of, based on the weight of the Chinese cabbage, about ⅕ by weight (w/w) to about 1/30 part by weight (w/w), preferably, about 1/10 part by weight (w/w) to about 1/20 part by weight (w/w), is dissolved in water, and the divided Chinese cabbage pieces are soaked in the salted water, and after being taken out of the salted water, salt was sprinkled on layers between the Chinese cabbage leaves, and the Chinese cabbages are salted for about 3 hours to about 8 hours, preferably, about 5 hours to about 6 hours, washed 2 times to 12 times, preferably, 3 times to 5 times, and placed on a large colander for dehydration;

(b) (second stages) a Chinese radish in an amount in a range of, based on the weight of the Chinese cabbages, about ½ part by weight (w/w) to about 1/10 part by weight (w/w), preferably, about ⅓ part by weight (w/w) to about ⅙ part by weight (w/w), is prepared, trimmed and washed after getting rid of radish leaves, and cut into thin strips in 3 cm to 6 cm long, and then, great green onions, chives, and mustard leaves each in an amount in a range of, based on the weight of the Chinese cabbages, about ⅕ part by weight (w/w) to about 1/30 part by weight (w/w), preferably, about 1/10 part by weight (w/w) to about 1/20 part by weight (w/w) are also trimmed and washed, and cut to the similar length with the Chinese radish strips;

(c) (third stage) garlic in an amount in a range of, based on the weight of the Chinese cabbages, about 1/50 part by weight (w/w) to about 1/300 part by weight (w/w), preferably, about 1/80 part by weight (w/w) to about 1/120 part by weight (w/w), ginger in an amount in a range of, based on the weight of the Chinese cabbages, about 1/100 part by weight (w/w) to 1/1000 part by weight (w/w), preferably, about 1/300 part by weight (w/w) to 1/600 part by weight (w/w), and salted shrimp in an amount in a range of, based on the weight of the Chinese cabbages, about 1/10 part by weight (w/w) to about 1/100 part by weight (w/w), preferably, about 1/20 part by weight (w/w) to about 1/40 part by weight (w/w) are finely minced, and anchovy sauce in an amount in a range of, based on the weight of the Chinese cabbages, about 1/10 part by weight (w/w) to about 1/100 part by weight (w/w), preferably, about 1/20 part by weight (w/w) to about 1/40 part by weight (w/w) is prepared;

(d) (fourth stage) glutinous rice in an amount in a range of, based on the weight of the Chinese cabbages, about 1/10 w/w to about 1/200 w/w, preferably, about 1/20 w/w to about 1/80 w/w is soaked in water, and rice porridge is cooked using the same, and after cooling the rice porridge, the salted shrimp, anchovy sauce, garlic, and ginger prepared in the third stage are added thereto with red pepper powder in an amount in a range of, based on the weight of the Chinese cabbages, about ½ w/w to about 1/40 w/w, preferably, about ⅕ w/w to about 1/10 w/w, and then, all the seasonings are mixed evenly;

(e) (fifth stage) after putting and mixing all the Chinese radish strips, great green onion, chives, and mustard leaves that are all cut to the similar length as in the second stage, kimchi seasoning is made by seasoning with salt in an amount in a range of, based on the weight of the Chinese cabbages, about ½ part by weight (w/w) to about 1/40 part by weight (w/w), preferably, about ⅕ part by weight (w/w) to about 1/10 part by weight (w/w), and sugar in an amount in a range of, based on the weight of the Chinese cabbages, about ½ part by weight (w/w) to about 1/40 part by weight (w/w), preferably, about ⅕ part by weight (w/w) to about 1/10 part by weight (w/w); and

(f) (sixth stage) after spreading the kimchi seasoning evenly between the Chinese cabbage leaves, each of the Chinese cabbages is rolled by the outermost leaf and placed one by one in a container in a way that the cross section of the Chinese cabbage faces up, and then, the container is stored in a low-temperature storage maintaining a temperature thereof between about 10° C. to about −10° C., preferably, between about 0° C. to about −2° C., so as to ripen the kimchi for about 3 months to about 5 years, preferably, about 6 months to about 2 years, thereby preparing the Chinese cabbage kimchi as a raw material;

Step 2: the raw materials for kimchi seasoning are inoculated onto an MRS medium, preferably, a modified MRS medium to which bromcresol purple and sodium azide are diluted with a peptone diluent and added, at a certain amount by a streak-plate method, thereby obtaining a medium inoculated with a strain;
Step 3: the strain-inoculated medium of Step 2 is cultured at a temperature in a range of about 27° C. to about 47° C., preferably, about 32° C. to about 39° C., for about 12 hours to about 72 hours, preferably, about 26 hours to about 52 hours, more preferably, about 33 hours to about 46 hours;
Step 4: the colonies of Step 3 are isolated purely from the MRS medium, preferable, the modified MRS medium to which bromocresol purple and sodium azide are diluted with a peptone diluent and added, thereby obtaining colonies that run yellow;
Step 5: the colonies of Step 4 are selected as tentative lactic acid bacteria; and
Step 6: the tentative strain selected in Step 5 is smeared on an MRS medium preferably an electrically-modified MRS medium, and cultured in an aerobic condition, thereby purely isolating a new Lactobacillus plantarum KC3 strain of the present disclosure having the following characteristics below.

As a result of identifying the strain purely isolated by the manufacturing process above, it is confirmed that the strain is a Gram-positive Bacillus, grows well regardless of the presence of oxygen, and is negative for catalase and motility. The strain is also found not to grow at a temperature between 15° C. and 45° C., and based on that no gas from glucose and no ammonia from arginine are produced, the strain is confirmed to belong to the genus Lactobacillus.

In addition, the nucleic sequence (SEQ ID NO: 1), which is obtained by collecting the colonies grown in the MRS medium and performing double-stranded DNA sequencing (Solgent, Korea) thereon, is searched by BLAST to identify the strain. As a result, the strain shows a homology of 99% to the Lactobacillus plantarum, confirming that the new microorganism of the present disclosure is the strain belonging to the Lactobacillus plantarum species.

The new Lactobacillus plantarum KC3 (hereinafter also referred to as “CKDB-KC3”) of the present disclosure is characterized by the following characteristics:

(1) Form of bacteria: Form of bacteria when cultured in MRS agar plate medium at 37° C. for 48 hours
\textcircled{1} Cell type: Bacillus
\textcircled{2} Mobility: None
\textcircled{3} Spore-forming ability: None
\textcircled{4} Gram staining: Positive

(2) Form of colony: Form of colonies when cultured in MRS agar plate medium at 37° C. for 48 hours
\textcircled{1} Shape: Round
\textcircled{2} Bulge: Convex
\textcircled{3} Surface: Smooth
\textcircled{4} Color: Milky-white

(3) Physiological properties
\textcircled{1} Temperature for growth and development
Temperature enable growth and development: 15° C. to 40° C.
Optimal temperature for growth and development: 36° C. to 38° C.
\textcircled{2} pH for growth and development
pH enable growth and development: 4.6 to 7.5
Optimal pH: 6.0 to 7.0
\textcircled{3} Effect on oxygen: Facultative anaerobic

(4) Catalase: Negative

(5) Gas generation: Negative

(6) Indole production: Negative

(7) Lactic acid production: Positive

(8) Biogenic amine production: Negative

Based on the results of identifying the microorganism and the bacteria characteristics above, the new strain isolated from kimchi was newly named Lactobacillus plantarum KC3 (also referred to as “CKDB-KC3”). Also, as described in the existing KR 10-2011883, the Lactobacillus plantarum KC3 was deposited at the Korea Research Institute of Bioscience and Biotechnology (Accession No: KCTC13375BP) on Oct. 20, 2017.

In addition, processes for a culture in the art, a concentrate of the culture, a dried material of the culture may be additionally performed (KR 10-1605516 disclosing “Method for Increasing Viability, Storage Stability, Acid Tolerance or Oxgall Tolerance of Lactic Acid Bacteria”).

The inventors of the present disclosure confirmed that the composition disclosed herein is useful as a pharmaceutical composition or a health functional food for ameliorating immune disorders and preventing and treating respiratory inflammatory diseases by confirming that the Lactobacillus plantarum KC3 strain exhibits new medical efficacy of activities especially on the amelioration of an immune disorder and the inhibition of a respiratory inflammatory disease through the following animal experiments targeting the Lactobacillus plantarum KC3 strain having an anti-obesity effect as disclosed in the existing KR 10-2011883: an experiment on the characteristics of the new lactic acid bacterium from the Lactobacillus plantarum KC3 (see Experimental Example 1); an experiment on the inhibitory effects of probiotics on the expression of inflammatory cytokines in the intestines (see Experimental Example 2); an experiment on the anti-inflammatory effect on ear edema (see Experimental Example 3); and an experiment on the defense effect against respiratory damage caused by air pollutants such as fine dust (see Experimental Example 4).

Accordingly, the present disclosure provides a pharmaceutical composition or a health functional food for ameliorating immune disorders and preventing and treating respiratory inflammatory diseases, the pharmaceutical composition or health functional food including, as an active ingredient, one or more selected from the group consisting of the Lactobacillus plantarum KC3 strain (also referred to as “CKDB-KC3”, Accession number: KCTC13375BP) obtained by the preparation method above, a culture thereof, a concentrate of the culture, and a dried material of the culture.

In addition, at least one selected from the group consisting of the Lactobacillus plantarum KC3 strain (also referred to as “CKDB-KC3”, Accession number: KCTC13375BP), the culture thereof, the concentrate of the culture, and the dried material of the culture have been eaten in the form of, for example, kimchi for a long time, and thus there is no problem such as toxicity and side effects.

The term “prevention” as used herein refers to all actions that can inhibit or delay inflammation, allergy, or asthma by administering a composition including the strain above. Also, the term “treatment” as used herein refers to all actions that can ameliorate or beneficially change symptoms of a disease by administering a composition including the strain.

As another aspect, the present disclosure provides a method of ameliorating an immune disorder and treating a respiratory inflammatory disease, the method including administering one or more selected from the group consisting of a Lactobacillus plantarum KC3 strain (also referred to as “CKDB-KC3”, Accession number: KCTC13375BP), a culture thereof, a concentrate of the culture, and a dried material of the culture, to a patient having an immune disorder and a respiratory inflammatory disease.

As another aspect, the present disclosure provides use of one or more selected from the group consisting of a Lactobacillus plantarum KC3 strain (also referred to as “CKDB-KC3”, Accession number: KCTC13375BP), a culture thereof, a concentrate of the culture, and a dried material of the culture, for the preparation of a therapeutic agent to treat a patient having an immune disorder and a respiratory inflammatory disease.

The pharmaceutical composition including the strain according to the present disclosure may be formulated in oral dosage form, such as a powder, granules, tablets, capsules, suspensions, emulsions, syrup, aerosol, and the like, in a suppository form, and in a sterilized injection solution form, each according to the methods in the art. Carriers, excipients, and diluents that may be included in the composition including the strain may include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In the case of formulation, a commonly used diluent or excipient, such as a filler, a weighting agent, a binder, a wetting agent, a disintegrant, a surfactant, and the like may be used for preparation. Examples of solid formulations for oral administration are a tablet, a pill, a powder, a granule, a capsule, and the like. Such a solid formulation may be prepared by mixing the strain and a fraction thereof with at least one excipient, such as starch, calcium carbonate, sucrose or lactose, gelatin, and the like. Also, in addition to a simple excipient, lubricants, such as magnesium stearate and talc, may be used. Examples of liquid formulations for oral administration are a suspension, an oral liquid, an emulsion, a syrup, and the like. In addition to water and liquid paraffin, which are simple diluents commonly used, various excipients, such as a wetting agent, a sweetening agent, a fragrance, a preservative, and the like may be included. Formulations for parenteral administration may include a sterile solution, a non-aqueous solvent, a suspension, an emulsion, a freeze-dried agent, a suppository, and the like.

For the formulations above, a non-aqueous solvent and a suspension, such as propylene glycol, polyethylene glycol, vegetable oil including olive oil, injectable ester including ethyl oleate, and the like may be used. As a base agent for the suppository, witepsol, macrogol, tween 61, cacao butter, laurin butter, glycerol, gelatin, or the like may be used.

A preferable dosage of the pharmaceutical composition including the strain of the present disclosure may vary according to conditions and a weight of a patient, severity of a disease, a drug form, and administration routes and periods, but may be appropriately selected by those skilled in the art. However, to obtain a desirable effect, the pharmaceutical composition including the strain of the present disclosure may be administered at a dosage in a range of about 0.0001 mg/kg to about 100 mg/kg, preferably, about 0.001 mg/kg to about 100 mg/kg, per day. The administration may be performed once a day or several times a day. The administration dosage does not limit the scope of the present disclosure in any aspect.

The strain may be administered to mammals, such as rats, mice, domestic animals, and humans, in various routes. All administration methods may be contemplated. For example, the administration may be performed orally or rectally or by an intravenous, intramuscular, subcutaneous, intrauterine dural, or intracerebroventricular injection.

The pharmaceutical composition of the present disclosure may include the strain in an amount in a range of about 0.1 wt % to about 50 wt % based on the total weight of the composition.

Formulations for parenteral administration may include a sterile aqueous solution, a non-aqueous solvent, a suspension, an emulsion, a freeze-dried agent, and a suppository. Examples of the non-aqueous solvent and the suspension may include propylene glycol, polyethylene glycol, vegetable oil such as olive oil, injectable ester such as ethyl oleate, and the like. As a base agent for the suppository, witepsol, macrogol, tween 61, cacao butter, laurin butter, glycerol, gelatin, or the like may be used.

A desirable dosage of the strain of the present disclosure may vary depending on conditions and a weight of a patient, severity of a disease, a drug form, administration routes and periods, but may be appropriately selected by those skilled in the art. However, to obtain a desirable effect, the strain may be administered at a dosage in a range of about 0.0001 mg/kg to about 100 mg/kg, preferably, about 0.001 mg/kg to about 100 mg/kg, once a day or several times a day. In the composition, the strain of the present disclosure may be mixed in an amount in a range of about 0.0001 wt % to about 50 wt % of the total weight of the composition.

The pharmaceutical composition of the present disclosure may be administered to mammals, such as rats, mice, domestic animals, and humans, in various routes. All administration methods may be contemplated. For example, the administration may be performed orally or rectally or by an intravenous, intramuscular, subcutaneous, intrauterine dural, and intracerebroventricular injection.

As another aspect, the present disclosure provides a probiotic agent for ameliorating an immune disorder and preventing or treating a respiratory inflammatory disease, the probiotic agent including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

As another aspect, the present disclosure provides a therapeutic method of ameliorating an immune disorder and treating a respiratory inflammatory disease, the therapeutic method including administering one or more selected from the group consisting of the Lactobacillus plantarum KC3 strain (also referred to as “CKDB-KC3”, Accession number: KCTC13375BP) according to the present disclosure, a culture thereof, a concentrate of the culture, and a dried material of the culture, to a patient having an immune disorder and a respiratory inflammatory disease.

As another aspect, the present disclosure provides use of one or more selected from the group consisting of the Lactobacillus plantarum KC3 strain (also referred to as “CKDB-KC3”, Accession number: KCTC13375BP), a culture thereof, a concentrate of the culture, and a dried material of the culture, for the preparation of a therapeutic agent to treat a patient having an immune disorder and a respiratory inflammatory disease.

In addition, the present disclosure may also provide a probiotic agent including, as an active ingredient, at least one selected from the group consisting of the Lactobacillus plantarum KC3 strain, a culture thereof, a concentrate of the culture, and a dried material of the culture.

The Lactobacillus plantarum KC3 strain of the present disclosure may be isolated from kimchi. The formulations may be prepared and administered in various types and methods according to methods known in the art. For example, the Lactobacillus plantarum KC3 strain of the present disclosure, the culture thereof, the concentrate of the culture, or the dried material of the culture may be prepared by mixing with a carrier commonly used in the pharmaceutical field, in the form of powder, liquids and solutions, tablets, capsules, syrup, suspensions, or granules for administration. The carrier may be, for example, a binder, a lubricant, a disintegrant, an excipient, a solubilizer, a dispersant, a stabilizer, a suspending agent, a colorant, a spice, and the like, but is not limited thereto. In addition, the administration dosage may be appropriately selected according to absorbance of an active ingredient in the body, an inactivation rate, an excretion rate, and age, gender, pedigree, and condition of a person to be administered, and severity of a disease.

As another aspect, the present disclosure provides a health functional food for ameliorating immune disorders and preventing or ameliorating respiratory inflammatory diseases, the health functional food including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

As another aspect, the present disclosure provides a health supplement food for ameliorating immune disorders and preventing or ameliorating respiratory inflammatory diseases, the health supplement food including, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

The term “health functional food” as defined herein refers to food prepared and processed by using a raw material or an ingredient having functionality useful to humans, according to the Law for Health Functional Foods 6727 in South Korea. Here, the term “functionality” indicates ingestion of food to adjust nutrients with regard to a structure and functions of the human body or to obtain effects advantageous to health care including physiological actions.

The health functional food for ameliorating immune disorders and preventing or ameliorating respiratory inflammatory diseases may include the strain in an amount in a range of about 0.01 wt % to about 95 wt %, preferably, about 1 wt % to about 80 wt %, based on the total weight of the composition.

In addition, for the purpose of ameliorating an immune disorder and preventing or ameliorating a respiratory inflammatory disease, the health functional food may be prepared and processed in the form of pharmaceutically acceptable administration, such as powder, a granule, a tablet, a capsule, a pill, a suspension, an emulsion, or a syrup, or in the form of a tea bag, a leached tea, a health beverage, or the like.

In addition, the present disclosure provides a food or a food additive for ameliorating an immune disorder and preventing or ameliorating a respiratory inflammatory disease, the food or the food additive including, as an active ingredient, one or more selected from the group consisting of the Lactobacillus plantarum KC3 strain (also referred to as “CKDB-KC3”, Accession number: KCTC13375BP), the culture thereof, the concentrate of the culture, and the dried material of the culture.

In addition, the health functional food may additionally include a food additive, and the suitability as a “food additives” is determined according to standards and criteria of items in accordance with the general provisions and general analytical method of the Korean Food Additive Code approved by the Ministry of Food and Drug Safety, unless otherwise specified.

Examples of products listed in the “Korean Food Additive Code” may include ketones, chemical products, such as glycine, potassium citrate, nicotinic acid, and cinnamic acid, natural additives, such as a persimmon color, licorice extract, crystalline cellulose and guar gum, and mixed formulations, such as monosodium L-glutamate, alkali agents for noodles, preservative formulation, and tar color formulation.

Examples of the functional food including the extract may include confectionary such as bread, rice cake, dried fruit, candy, chocolate, chewing gum, and jam, ice cream products such as ice cream, frozen dessert, and ice cream powder, dairy products such as milk, low-fat milk, lactose-free milk, processed milk, goad milk, fermented milk, buttermilk, condensed milk, milk cream, butter oil, butter oil, natural cheese, processed cheese, milk powder, and whey, meat products such as processed meat products, egg products, and hamburger, fish and meat products including processed fish and meat products such as fish cake, ham, sausage, and bacon, noodles such as instant noodle, dried noodle, raw noodle, instant fried noodle, instant non-fried noodle, processed noodle, frozen noodle, pasta, fruit drink, vegetable drink, carbonated drink, soy milk, lactic acid beverage such as yogurt, beverage such as mixed drink, seasonings such as soy sauce, soybean paste, red pepper paste, black soybean paste, fermented soybean paste, mixed soy paste, vinegar, sauces, tomato ketchup, curry, and dressing, margarine, shortening, and pizza. However, embodiments of the present disclosure are not limited thereto.

The health functional beverage composition of the present disclosure has no particular limitation on components other than the inclusion of the strain above as an essential ingredient at an indicated ratio, and may additionally include various flavorings or natural carbohydrates as in the existing beverages. Examples of the above natural carbohydrates include general sugar such as monosaccharides (e.g., glucose, fructose, etc.), disaccharides (e.g., maltose, sucrose, etc.), and polysaccharides (e.g., dextrin, cyclodextrin, etc.), and sugar alcohols such as xylitol, sorbitol, and erythritol. In addition to those described above, natural flavorings (thaumatin, Stevia extracts (e.g., rebaudioside A, glycyrrhizin, etc.)), and synthetic flavoring agents (e.g., saccharin, aspartame, etc.) may be advantageously used as flavorings. A ratio of the natural carbohydrates may generally range from about 1 g to about 20 g, preferably, from about 5 g to about 12 g, per 100 ml of the composition.

In addition to those described above, the composition may contain various nutrients, a vitamin, a mineral (an electrolyte), flavorings such as synthetic and natural flavorings, a coloring agent and an improving agent (cheese, chocolate, etc.), pectic acid and the salt thereof, alginic acid and the salt thereof, organic acid, a protective colloidal adhesive, a pH regulator, a stabilizer, a preservative, glycerin, alcohol, a carbonizing agent used in a carbonate beverage, and the like. The other components than the aforementioned components may be fruit pulp for preparing natural fruit juice, a fruit juice beverage, and vegetable juice. Such components may be used independently or in combination. A ratio of the additives is not so important, but is generally selected in a range of about 0 part by weight to about 20 parts by weight based on 100 parts by weight of the composition of the present disclosure.

Also, the strain of the present disclosure may be added to food or beverages for prevention of purposed diseases. In this case, an amount of the strain in food or beverages may be in a range of about 0.01 wt % to about 15 wt % based on the total weight of the food, and may be in a range of about 0.02 g to about 5 g, preferably, about 0.3 g and 1 g based on 100 ml of the health beverage composition.

During a process of preparing the health functional food, the strain of the present disclosure added to food including beverages may be appropriately adjusted according to necessity.

### Advantageous Effects of Disclosure

By confirming that the Lactobacillus plantarum KC3 strain exhibits excellent activities of ameliorating an immune disorder and inhibiting a respiratory inflammatory disease through the following animal experiments targeting the Lactobacillus plantarum KC3 strain having an anti-obesity effect as disclosed in the existing KR 10-2011883: an experiment on the characteristics of the new lactic acid bacteria from the Lactobacillus plantarum KC3 (see Experimental Example 1); an experiment on the inhibitory effects of probiotics on the expression of inflammatory cytokines in the intestines (see Experimental Example 2); an experiment on the anti-inflammatory effect on ear edema (see Experimental Example 3); and an experiment on the defense effect against respiratory damage caused by air pollutants such as fine dust (see Experimental Example 4), the confirmed is useful as a pharmaceutical composition or a health functional food for ameliorating immune disorders and preventing and treating respiratory inflammatory diseases. Accordingly, the composition can be widely utilized as a therapeutic agent for the amelioration of an immune disorder and the prevention and treatment of a respiratory inflammatory disease.

### BRIEF DESCRIPTION OF DRAWINGS

FIG. 1 shows results of an experiment on the effects on a ratio of neutrophils to the total number of cells in bronchoalveolar lavage (BAL) fluid;

FIG. 2 shows a deposit certificate of Lactobacillus plantarum KC3;

FIG. 3 shows results of an experiment on bile tolerance against the Lactobacillus plantarum KC3 strain of the present disclosure (wherein all numerical values (or data) are the mean±standard deviation of 3 repetitions, and * represents a case of p<0.05 between with oxgall and without oxgall); and

FIG. 4 shows results of an experiment on pH resistance against the L Lactobacillus plantarum KC3 strain of the present disclosure.

### BEST MODE

It would be obvious to one of ordinary skill in the art that various modifications and variations may be made for compositions, use, and preparation of the present disclosure without departing from the spirit or scope of the present disclosure.

One or more embodiments of the present disclosure are described in detail, but it should be understood that the present disclosure is not limited to those embodiments in any manner.

However, embodiments and experimental examples below are merely examples without limiting the scope of the present disclosure, and the present disclosure is not limited to those embodiments and experimental examples.

### Example 1. Isolation of New Lactic Acid Bacteria from Lactobacillus plantarum KC3

1-1. Preparation of Raw Materials of Kimchi

For use as cabbage kimchi which is a raw material of the present disclosure, kimchi for family use in North Jeolla Province was prepared according to the following process using materials all purchased from a local mart (Hanaro Mart, Wansan-gu, Jeonju-si).

(1) Step 1: Five Chinese cabbages (about 1 kg each) were prepared and cut into two pieces after getting rid of inedible portions. 500 g of salt was dissolved in water in a container for salting. The divided Chinese cabbage pieces were soaked in salted water, and after being taken out of the salted water, salt was sprinkled in layers between cabbage leaves. The Chinese cabbages were then salted for 5 to 6 hours, washed with clean water 3 to 4 times, and placed on a large colander for dehydration.

(2) Step 2: Two Chinese radishes (about 1.2 kg each) were prepared, trimmed and washed after getting rid of radish leaves, and then cut into thin strips in 4-5 cm long. Half bundle of great green onions (about 0.5 kg), half bundle of chives (about 0.5 kg), and half bundle of mustard leaves (about 0.5 kg) were also trimmed and washed, and cut into the same length as the radish strips.

(3) Step 3: 50 g of garlic, 10 g of ginger, and 200 g of salted shrimp were finely minced, and 300 ml (about 200 g) of anchovy sauce was prepared. Rice porridge was cooked with 150 g of glutinous rice soaked in water. After cooling the rice porridge, the prepared anchovy sauce and the minced salted shrimp, garlic, and ginger were added thereto with 500 g of red pepper powder, and all the seasonings were mixed evenly.

(4) Step 4: After putting and mixing all the radish strips, great green onion, chives, and mustard leaves that were all cut to the similar length as in Step 2, kimchi seasoning was made by seasoning with salt (about 0.5 kg) and sugar (about 0.5 kg).

(5) Step 5: After spreading the kimchi seasoning evenly between the Chinese cabbage leaves, the Chinese cabbage was rolled by the outermost leaf. Then, the Chinese cabbage was placed one by one in a container in a way that the cross section of the Chinese cabbage faced up, and the container was stored in a low-temperature storage (0° C. to −2° C.) so as to ripen the kimchi for 1 year, thereby producing raw materials for the Chinese cabbage kimchi.

1-2. Isolation of New Microorganism

Regarding isolation and identification processes for the Lactobacillus plantarum KC3 strain of the present disclosure, the raw materials for the Chinese cabbage kimchi of Section 1-1 were inoculated by 0.1 ml each onto an MRS sodium medium (supplemented with MRS medium (DF0881-17-5, Difco) and 1.5% agar (214010, Difco)) to which bromocresol purple (114375, Sigma) and sodium azide (S2002, sigma) were diluted with a peptone diluent (MB-B2220, MB cell) and added, by a streak-plate method. After culturing in an anaerobic condition at 37° C. for 48 hours, colonies that turned yellow in the medium were selected as tentative lactic acid bacteria.

As a result of identifying the isolated strain, it was confirmed that the strain was a gram-positive facultative anaerobic Bacillus was negative for the catalase and motility.

It was also confirmed that the strain did not grow at 15° C. and 45° C., and based on that no gas from glucose and no ammonia from arginine were produced, the strain was confirmed to belong to the genus Lactobacillus.

1-3. Identification of Microorganism (Based on Analysis of Glucose Utilization and 16s rRNA Identification)

1-3-1. Analysis of Glucose Utilization

The glucose utilization of the selected lactic acid bacteria was analyzed using an API CHL50 kit (50300, bioMerieux). As a result of the analysis, it was confirmed that, as shown in Table 1 below, glucose from D-ribose, D-galactose, D-glucose, D-fructose, D-mannose, D-mannitol, D-sorbitol, methyl-αD-mannopyranoside, amyglandine, albutine, esculin ferric citrate, D-cellobiose, D-maltose, D-lactose, D-melibiose, D-saccharose, D-trehalose, D-melezitose, and D-raffinose was utilized.

TABLE 1

| Glucose | Utilization | Glucose | Utilization |
|---|---|---|---|
| Control | − | Esculin ferric | + |
| | | citrate | |
| Glycerol | − | Salicin | ± |
| Erythritol | − | D-Celiobiose | + |
| D-Arabinose | − | D-Maltose | + |
| L-Arabinose | − | D-Lactose | + |
| D-Ribose | + | D-Melibiose | + |
| D-Xylose | − | D-Saccharose | + |
| L-Xylose | − | D-Trehalose | + |
| D-Adonitol | − | Inulin | − |
| Methyl-βD- | − | D-Melezitose | + |
| Xylopyranoside | | | |
| D-Galactose | + | D-Raffinose | + |
| D-Glucose | + | Amidon | − |
| D-Fructose | + | Glycogen | − |
| D-Mannose | + | Xylitol | − |
| L-Sorbose | − | Gentiobiose | ± |
| L-Rhamnose | − | D-Turanose | − |
| Dulcitol | − | D-Lyxose | − |
| Inositol | − | D-Tagatose | − |
| D-Mannitol | + | D-Fucose | − |
| D-Sorbitol | + | L-Fucose | − |
| Methyl-αD- | + | D-Arabitol | − |
| Mannopyranoside | | | |
| Methyl-αD- | − | L-Arabitol | − |
| Glucopyranoside | | | |
| N-AcetylGlucosamine | ± | potassium Gluconate | − |
| Amygdalin | + | potassium | − |
| | | 2-KetoGluconate | |
| Arbutin | + | potassium | − |
| | | 5-KetoGluconate | |

1-3-2. 16s rRNA Identification

The colonies grown on the MRS solid medium (supplemented with MRS medium (DF0881-17-5, Difco) and 1.5% agar (214010, Difco)) were collected and subjected to double-stranded DNA sequencing (Solgent, Korea). As a result of identifying the strain by BLAST, the obtained nucleic acid sequence (SEQ ID NO: 1 in Table 2) showed a homology of 99% to the Lactobacillus plantarum, confirming that the new microorganism of the present disclosure was the strain (hereinafter also referred to as “new lactic acid bacteria from KC3” or “CKDB-KC3”).

TABLE 2
16s rRNA nucleic sequence of
Lactobacillus plantarum KC3

| | SEQ |
|---|---|
| 16s rRNA nucleic sequence of | ID |
| Lactobacillus plantarum KC3 | NO. |
| TATGGCTCAGGACGAACGCTGGCGGCGTGC | 1 |
| CTAATACATGCAAGTCGAACGAACTCTGGT | |
| ATTGATTGGTGCTTGCATCATGATTTACAT | |
| TTGAGTGAGTGGCGAACTGGTGAGTAACAC | |
| GTGGGAAACCTGCCCAGAAGCGGGGGATAA | |
| CACCTGGAAACAGATGCTAATACCGCATAA | |
| CAACTTGGACCGCATGGTCCGAGTTTGAAA | |
| GATGGCTTCGGCTATCACTTTTGGATGGTC | |
| CCGCGGCGTATTAGCTAGATGG | |
| TGGGGTAACGGCTCACCATGGCAATGATAC | |
| GTAGCCGACCTGAGAGGGTAATCGGCCACA | |
| TTGGGACTGAGACACGGCCCAAACTCCTAC | |
| GGGAGGCAGCAGTAGGGAATCTTCCACAAT | |
| GGACGAAAGTCTGATGGAGCAACGCCGCGT | |
| GAGTGAAGAAGGGTTTCGGCTCGTAAAACT | |
| CTGTTGTTAAAGAAGAACATATCTGAGAGT | |
| AACTGTTCAGGTATTGACGGTATTTAACCA | |
| GAAAGCCACGGCTAACTACGTGCCAGCAGC | |
| CGCGGTAATACGTAGGTGGCAAGCGTTGTC | |
| CGGATTTATTGGGCGTAAAGCGAGCGCAGG | |
| CGGTTTTTTAAGTCTGATGTGAAAGCCTTC | |
| GGCTCAACCGAAGAAGTGCATCGGAAACTG | |
| GGAAACTTGAGTGCAGAAGAGGACAGTGGA | |
| ACTCCATGTGTAGCGGTGAAATGCGTAGAT | |
| ATATGGAAGAACACCAGTGGCGAAGGCGGC | |
| TGTCTGGTCTGTAACTGACGCTGAGGCTCG | |
| AAAGTAT | |
| GGGTAGCAAACAGGATTAGATACCCTGGTA | 1 |
| GTCCATACCGTAAACGATGAATGCTAAGTG | |
| TTGGAGGGTTTCCGCCCTTCAGTGCTGCAG | |
| CTAACGCATTAAGCATTCCGCCTGGGGAGT | |
| ACGGCCGCAAGGCTGAAACTCAAAGGAATT | |
| GACGGGGGCCCGCACAAGCGGTGGAGCATG | |
| TGGTTTAATTCGAAGCTACGCGAAGAACCT | |
| TACCAGGTCTTGACATACTATGCAAATCTA | |
| AGAGATTAGACGTTCCCTTCGGGGACATGG | |
| ATACAGGTGGTGCATGGTTGTCGTCAGCTC | |
| GTGTCGTGAGATGTTGGGTTAAGTCCCGCA | |
| ACGAGCGCAACCCTTATTATCAGTTGCCAG | |
| CATTAAGTTGGGCACTCTGGTGAGACTGCC | |
| GGTGACAAACCGGAGGAAGGTGGGGATGAC | |
| GTCAAATCATCATGCCCCTTATGACCTGGG | |
| CTACACACGTGCTACAATGGATGGTACAAC | |
| GAGTTGCGAACTCGCGAGAGTAAGCTAATC | |
| TCTTAAAGCCATTCTCAGTTCGGATTGTAG | |
| GCTGCAACTCGCCTACATGAAGTCGGAATC | |
| GCTAGTAATCGCGGATCAGCATGCCGCGGT | |
| GAATACGTTCCCGGGCCTTGTACACACCGC | |
| CCGTCACACCATGAGAGTTTGTAACACCCA | |
| AAGTCGGTGGGGTAACCTTTTAGGAACCAG | |
| CCGCCTAAGGTGGGACAGATGATTAGGGTG | |
| AAGTCGTACA | |

1-3-3. Characteristics of Microorganism

The characteristics of the new Lactobacillus plantarum KC3 according to the present disclosure are as follows:

(1) Form of bacteria
Form of bacteria when cultured in MRS agar plate medium at 37° C. for 48 hours
\textcircled{1} Cell type: Bacillus
\textcircled{2} Mobility: None
\textcircled{3} Spore-forming ability: None
\textcircled{4} Gram staining: Positive

(2) Shape of colony
Form of colonies when cultured in MRS agar plate medium at 37° C. for 48 hours
\textcircled{1} Shape: Round
\textcircled{2} Bulge: Convex
\textcircled{3} Surface: Smooth
\textcircled{4} Color: Milky-white

(3) Physiological properties
\textcircled{1} Temperature for growth and development
Temperature enable growth and development: 15° C. to 40° C.
Optimal temperature for growth and development: 36° C. to 38° C.
\textcircled{2} pH for growth and development
pH enable growth and development: 4.6 to 7.5
Optimal pH: 6.0 to 7.0
\textcircled{3} Effect on oxygen: Facultative anaerobic

(4) Catalase: Negative

(5) Gas generation: Negative

(6) Indole production: Negative

(7) Lactic acid production: Positive

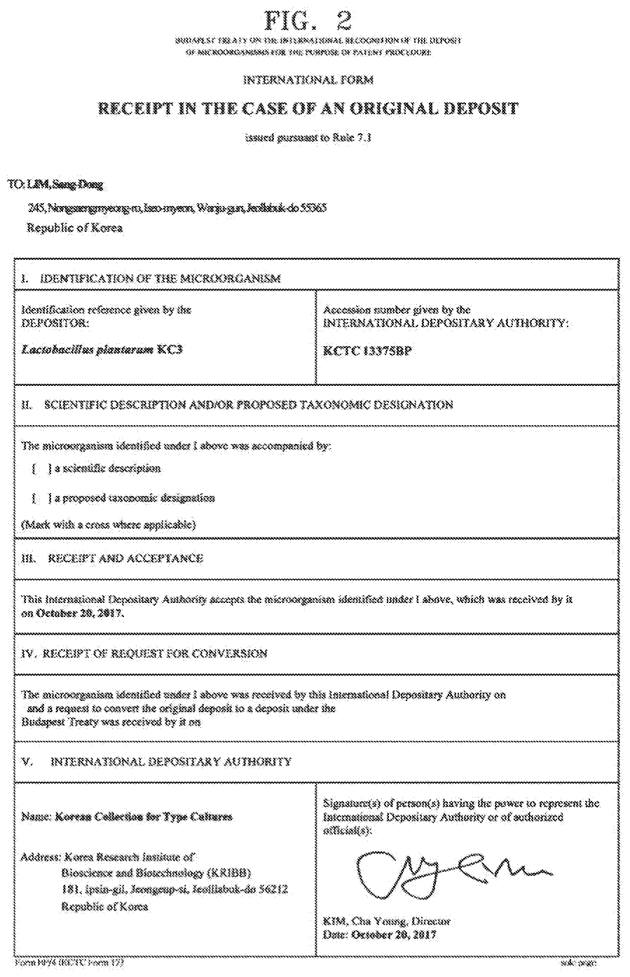

(8) Biogenic amine production: Negative
Based on the results of the microorganism identification and the bacteria characteristics above, a new strain isolated from kimchi was named Lactobacillus plantarum KC3 and deposited at the Korea Research Institute of Bioscience and Biotechnology (Accession No: KCTC13375BP) on Oct. 20, 2017 (see FIG. 2).

### Experimental Example 1. Characteristics of New Lactic Acid Bacteria from Lactobacillus plantarum KC3

1-1. Tolerance Experiment of Stomach Acid and Bile Acid

Gastric acid secretion in gastric fluids and bile acid secreted from the pancreas are significantly important factors affecting the survival of microorganisms. Thus, in order to confirm gastric acid-resistance and bile acid-resistance of the new lactic acid bacteria from the Lactobacillus plantarum KC3 of the present disclosure, an experiment was performed as follows by applying the method described in the document. (Kararli, T T, Comparison of the gastrointestinal anatomy, physiology, and biochemistry of humans and commonly used laboratory animals. Biopharm Drug Dispos 1995 16(5):351-380. doi: 10.1002/bdd.2510160502)

It was a process of examining the resistance to artificial gastric fluids and bile to explore the possibility for use as probiotics, and then selecting and identifying strains having excellent activity and strong resistance.

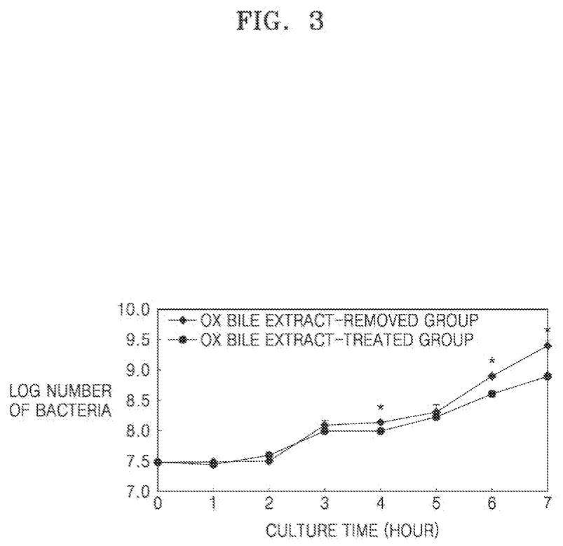

FIG. 3 is a diagram showing the results of the bile-resistance test against the L. plantarum KC3 strain of the present disclosure.

More specifically, the Lactobacillus plantarum KC3 strain was grown in an MRS medium (with oxgall) containing 0.03% of bile (oxgall) and 0.05% of L-cysteine and an MRS medium (without oxgall) containing 0.05% of L-cysteine. All values (or data) are the mean±standard deviation for triplicate experiments, and * indicates a case where p<0.05 between with oxgall and without oxgall.

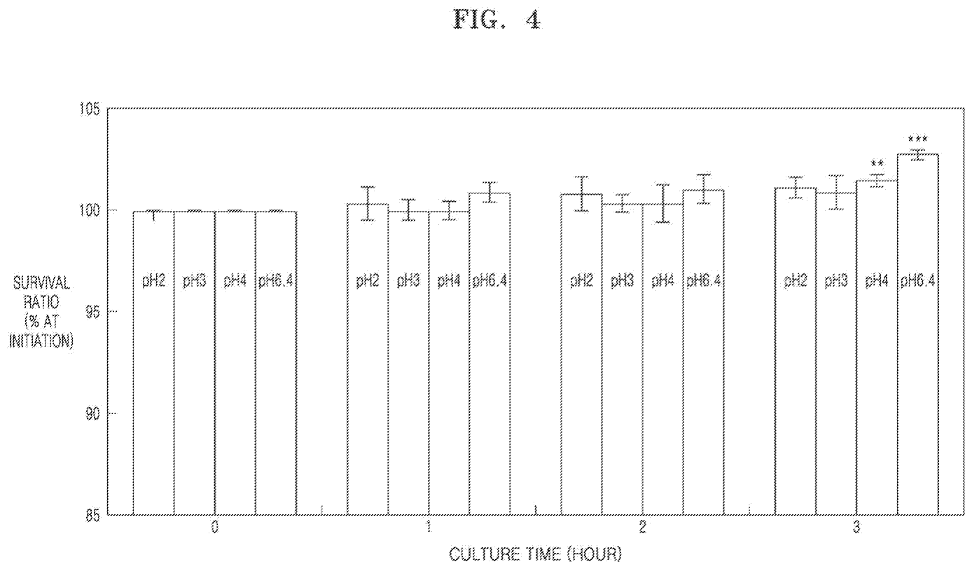

FIG. 4 is a diagram showing the results of pH resistance test against the Lactobacillus plantarum KC3 strain of the present disclosure.

More specifically, the figure shows the survival rate of the Lactobacillus plantarum KC3 strain after 3 hours in hydrochloric acid solution having a pH of 2.0, 3.0, 4.0 and 6.4, and as compared with the start point (or start time), * indicates a case of p<0.05, ** indicates a case of p<0.01, and *** indicates a case of p<0.001.

1-2. Antibacterial Activity Experiment

In order to confirm antibacterial activity of the new lactic acid bacteria from the Lactobacillus plantarum KC3, an antibacterial activity experiment was performed.

The antibacterial activity experiment was to confirm the inhibitory activity against Escherichia coli, Salmonella typhimurium, Staphylococcus aureus, and Listeria monocytogenes. The antibacterial activity was considered better when having stronger inhibitory activity against harmful bacterial.

Table 3 shows the experimental results about the antibacterial activity of the Lactobacillus plantarum KC3 strain, wherein the initial number of bacteria of the Lactobacillus plantarum KC3 strain was about 2.10±0.17×10⁶ CFU/mL, and the results were obtained after 6 hours of the experiments at 37° C. Here, all values (or data) are the mean±standard deviation for triplicate experiments.

TABLE 3
Antibacterial activity of Lactobacillus plantarum KC3

| | Growth | | | | |
|---|---|---|---|---|---|
| | Pathogens | | KC3 + pathogens^{a} | | Inhibi- |
| Pathogens | CFU/mL | pH | CFU/mL | pH | tion (%) |
| Escherichia | 3.23 ± 0.25 × | 5.98 | 8.50 ± 0.05 × | 4.84 | 73.98% |
| coli | 10⁶ | | 10⁵ | | |
| Salmonella | 6.46 ± 0.35 × | 6.10 | 4.00 ± 0.26 × | 5.25 | 38.14% |
| Typhimurium | 10⁶ | | 10⁶ | | |
| Listeria | 1.57 ± 0.20 × | 6.06 | 1.13 ± 0.06 × | 4.94 | 27.97% |
| monocytogenes | 10⁵ | | 10⁵ | | |
| Staphyloccous | 3.46 ± 0.87 × | 6.08 | 2.83 ± 0.61 × | 4.9 | 18.27% |
| aureus | 10⁶ | | 10⁶ | | |

1-3. Antibiotics Resistance Experiment

In order to confirm antibiotics resistance level of the new lactic acid bacteria from the of the Lactobacillus plantarum KC3 the present disclosure, an experiment was performed as follows by applying the method described in the document ([1] Mathur, S. and R. Singh, Antibiotic resistance in food lactic acid bacteria—A review. Int. J. Food Microbiol 2005 105: 281-295); [2] European Food Safety Authority, Guidance on the assessment of bacterial susceptibility to antimicrobials of human and veterinary importance: EFSA Panel on Additives and Products or Substances used in Animal Feed (FEEDAP). The EFSA Journal 2012 2740:1-10. doi:10.2903/j.efsa.2012.2740).

To measure the antibiotics resistance level of the strain, an MIC test was carried out. Lactic acid bacteria that were inoculated into an MRS medium (DF0881-17-5, Difco) and cultured at 37° C. for 18 hours were spread on an LSM solid medium (90% iso-sensitest broth (CM0473, Oxoid), 10% MRS medium (DF0881-17-5, Difco), and 1.5% agar (214010, Difco)). Strips for each type of antibiotics, such as Amikacin (92018, Liofilchem srl), Gentamycin (92009, Liofilchem srl), Kanamycin (92034, Liofilchem srl), Streptomycin (92112, Liofilchem srl), Penicillin-G (92102, Liofilchem srl), Oxacillin (92015, Liofilchem srl), Ampicillin (920030, Liofilchem srl), Bacitracin (92019, Liofilchem srl), Rifampicin (92001, Liofilchem srl), Polymyxin B (92004, Liofilchem srl), Chloramphenico I (92075, Liofilchem srl), Vancomycin (92057, Liofilchem srl), and the like, were put on the medium, and the bacteria were grown at 37° C. for 24 hours. Then, a section where a clear zone disappeared was observed with the naked eyes, and an MIC was measured therefrom.

Table 4 shows the results of the antibiotics resistance of the Lactobacillus plantarum KC3 strain. In Table 4, R indicates resistance and represents that the size of an inhibition zone is about 0 mm; IS indicates medium resistance and represents that the size of an inhibition zone is in a range of about 1 mm to about 5 mm; and S indicates resistance and represents that the size of an inhibition zone is greater than about 5 mm.

TABLE 4
Antibiotics resistance of Lactobacillus plantarum KC3

| Anti-microbial | Antibiotic | | Anti-microbial | Antibiotic | |
|---|---|---|---|---|---|
| agents | resistance | | agents | resistance | |
| Aminoglycosides | | | Gram-positive- | IS | (4 mm) |
| | | | spectrum | | |
| Amikacin | IS | (1 mm) | Bacitracin | S | (7 mm) |
| Gentamycin | IS | (3 mm) | Rifampicin | S | (7 mm) |
| Kanamycin | R | (0 mm) | Novabiocin | S | (7 mm) |
| Neomycin | IS | (3 mm) | Lincomycin | S | (10 mm) |
| Streptomycin | R | (0 mm) | Gram-negative | | |
| | | | spectrum | | |
| β-lactams | | | Polymyxin B | R | (0 mm) |
| Penicillin-G | IS | (5 mm) | Broad spectrum | | |
| Oxacillin | IS | (2 mm) | Chloramphenicol | S | (10 mm) |
| Ampicillin | S | (14 mm) | Vancomycin | R | (0 mm) |

1-4. Biogenic Amine Producibility Experiment

To confirm biogenic amine producibility of the new lactic acid bacteria from the Lactobacillus plantarum KC3 of the present disclosure, an experiment was performed as follows by applying the method described in the document (Bover-Cid S, Holzapfel W H, Improved screening procedure for biogenic amine production by lactic acid bacteria. Int J Food Microbiol 1999 53:33-41. doi:10.1016/S0168-1605(99)00152-X).

Biogenic amines are produced by fermentation of food and may vary depending on a type of microorganisms or chemical and physical conditions. Since biogenic amines produced in fermented food can cause food poisoning or allergic reactions, the biogenic amines are considered as important criteria for selecting a safe strain for food engineering ([1] Ladero V et al., Toxicological effects of dietary biogenic amines. Curr Nutr Food Sci. 2010 6:145-156. 10.2174/157340110791233256; [2] European Food Safety Authority, Scientific Opinion on risk-based control of biogenic amine formation in fermented foods: EFSA Panel on Biological Hazards. The EFSA Journal 2011 9:1-93. doi:10.2903/j.efsa.2011.2393).

In this regard, to confirm whether the strain of the present disclosure has formed the biogenic amines, the strain grown in an MRS liquid medium (DF0881-17-5, Difco) at 37° C. for 6 hours was transferred to a specialized medium as described in the document (Bover-Cid S, Holzapfel W H, Improved screening procedure for biogenic amine production by lactic acid bacteria. Int J Food Microbiol 1999 53:33-41. doi:10.1016/S0168-1605(99)00152-X), and then, cultured at 37° C. for 48 hours.

An MRS liquid medium (DF0881-17-5, Difco) to which an amino acid precursor for each of tyrosine (SIGMA, T1145), histidine (SIGMA, H5659), ornithine (SIGMA, O2375), and lysine (DAEJUNG, 5093-4105) was added was prepared. In each medium, it was confirmed whether the biogenic amines, i.e., tyramine, histamine, putrescine, and cadaverine, were produced by the strain. In detail, into an MRS liquid medium (DF0881-17-5, Difco) to which 0.1% of the amino acid precursor was added, 1% of the isolated Lactobacillus plantarum strain was, and then sub-cultured 5 times to 10 times.

The resulting strain was then spread on a biogenic amine identification medium [prepared by mixing 0.5% of tryptone, 0.5% of yeast extract, 0.5% of cocoon extract, 0.5% of sodium chloride, 0.25% of glucose, 0.05% of tween-80, 0.02% of magnesium sulfate, 0.005% of manganese sulfate, 0.004% of iron sulfate, 0.2% of citric acid salt, 0.001% of thiamine, 0.2% of K₂PO₄, 0.01% of calcium carbonate, 0.005% of pyridoxal-5-phosphate, 1% of amino acid, 0.006% of bromocresol purple, and 2% of agar with distilled water and adjusting the pH to 5.3 for use], and cultured at 37° C. for 24 hours to 48 hours. Then, by checking whether the color changes to purple, the biogenic amine producibility of the strain was determined.

Bromocresol purple contained in a decarboxylase medium is yellow at pH 5.2, but turns purple as the pH increases to 6.8. Thus, based on the color that turns purple as the pH increases by the production of the biogenic amines, the production of the biogenic amines was confirmed.

Table 5 below shows the results of analyzing the biogenic amine producibility of the Lactobacillus plantarum KC3 strain. As shown in Table 4, it was confirmed that the strain was negative for all of putrescine, tyramine, histamine, and cadaverine. Accordingly, it was confirmed that the strain of the present invention had no ability to produce the biogenic amines that can induce hypersensitive immune responses.

TABLE 5
Biogenic amine producibility of Lactobacillus plantarum KC3

Biogenic amines

| Strain | Putrescine | Tyramine | Histamine | Cadaverine |
|---|---|---|---|---|
| KC3 | — | — | — | — |

### Experimental Example 2: Inhibitory Effect of Probiotics on Expression of Intestinal Inflammatory Cytokines

In order to confirm whether the probiotics Lactobacillus plantarum KC3 (KC3) inhibits mRNA expression of an inflammatory cytokine induced by LPS, RT-PCR was performed as follows by applying the method described in the document. (Verma N et al., Profiling of ABC transporters during active ulcerative colitis and in vitro effect of inflammatory modulators. Dig Dis Sci. 2013 August; 58(8):2282-92. doi: 10.1007/s10620-013-2636-7; [2] Bhattacharyya S et al., Lipopolysaccharide activates NF-kappaB by TLR4-Bcl10-dependent and independent pathways in colonic epithelial cells. Am J Physiol Gastrointest Liver Physiol. 2008 October; 295(4):G784-90. doi: 10.1152/ajpgi.90434.2008.])

2-1. Experimental Method

HT-29 cells (KCBL No. 30038, large intestinal epithelial cell line, Human) were purchased from the Korean Cell Line Bank (KCLB, Seoul, Korea), and inoculated into a 6-well cell plate at a concentration of 1×10⁵ cell/mL and cultured in a cell incubator (Panasonic MCO-18AC-PK; under conditions of 37° C., 5% CO₂) until a monolayer was formed.

Samples for Example spread on an MRS solid medium (containing MRS medium (DF0881-17-5, Difco) and 1.5% agar (214010, Difco)) which is a freezing stock. After culturing 37° C. for 24 hours, colonies that were confirmed to be purely isolated were collected with a platinum loop and cultured in an MRS liquid medium (DF0881-17-5, Difco) at 37° C. for 18 hours to 24 hours.

The culture medium that has been completely cultured was subjected to centrifugation to remove the supernatant, and then, was washed with PBS twice to obtain bacterial cells only. The bacterial cells were diluted at a concentration of 1×10⁸ CFU/mL in an antibiotics-free and FBS-free DMEM cell culture medium (GenDEPOT, CM003-050). When the cells reached confluency, 20 μl of each bacterial suspension was dispensed into the cells and cultured in a cell incubator (Panasonic MCO-18AC-PK; under conditions of 37° C. and 5% CO₂). Afterwards, a washing process was performed thereon twice, each using PBS, and an LPS-containing cell medium was dispensed thereinto and allowed for a reaction for 16 hours.

After completion of the reaction, cells were detached by treatment with trypsin-EDTA (Gibco, 25200056). Then, RNA was extracted from the cells by using a commercially available kit (Promega, Z6010), and cDNA was synthesized.

Next, mRNA expression levels of inflammatory cytokines were confirmed through RT-PCR. The genomic nucleic sequences used for PCR analysis are listed in Table 6.

TABLE 6
Genomic nucleic sequence for PCR analysis

| | | | Se- |
|---|---|---|---|
| Target | | | quence |
| gene | Primer | Sequences | I.D. |
| β-actin | Forward | 5′-AGGTGAAGGTCGGAGTCAACG-3′ | 2 |
| | Reverse | 5′-GCTCCTGGAAGATGGTGATGG-3′ | 3 |
| IL-1β | Forward | 5′-ACAGATGAAGTGCTCCTTCCA-3′ | 4 |
| | Reverse | 5′-GTCGGAGATTCGTAGCTGGAT-3′ | 5 |
| TNFa | Forward | 5′-GAAAGCATGATCCGGGACGTG-3′ | 6 |
| | Reverse | 5′-GATGGCAGAGAGGAGGTTGAC-3′ | 7 |

See, Jinsil Choo. (2016) A study on the role of high-fat diet inducec Fabp6 in colon cancer cell growth, (Masters dissertation). Sookmyung Women's University, Seoul, Republic of Korea; Li J et al., Regulation of IL-8 and IL-1β expression in Crohn's disease associated NOD2/CARD15 mutations. Hum Mol Genet. 2004 Aug. 15; 13(16): 1715-25; Hossen MJ et al., Thymoquinone: An IRAKI inhibitor with in vivo and in vitro anti-inflammatory activities. Sci Rep. 2017 Feb. 20; 7:42995. doi: 10.1038/srep42995.

2-2. Experiment Results

As a result of the experiment above, it was confirmed as shown that the probiotics Lactobacillus plantarum KC3 (KC3) as in the example of the present disclosure exhibited effects of significantly inhibiting the expression of inflammatory cytokines (IL-1β and TNFα) that were increased by LPS using the HT-29 cell line (see Table 7)

TABLE 7
Inhibitory effect on expression of cytokines

| | IL-1β | TNFα |
|---|---|---|
| Control | 0.068 ± 0.006 | 0.033 ± 0.005 |
| LPS | 1.015 ± 0.248 | 1.000 ± 0.032 |
| LGG | 0.758 ± 0.147 | 1.223 ± 0.052 |
| KC3 | 0.438 ± 0.005 | 0.216 ± 0.124 |

LGG: Lactobacillus rhamnosus GG (positive control group)

### Experimental Example 3: Anti-Inflammatory Effect Test on Ear Edema

To confirm anti-inflammatory effect of the probiotic lactic acid bacteria (Lactobacillus plantarum KC3, KC3) on ear edema, an experiment was performed according to the croton oil-induced mouse ear edema method by applying the method described in the document (Dong Wook Kim, Kun Ho Son, Hyeun Wook Chang, KiHwan Bae and Sam Sik Kang, Arch Pharm Res., 2003, 3, pp 232-236).

3-1. Experimental Method

To evaluate anti-inflammatory activity of the samples of the Example above, a croton oil-induced mouse ear edema test was carried out, and results thereof were obtained by measuring the ear thickness of the samples as shown in Table 8. Croton oil causes inflammation generating rubefaction, swelling, and blisters when applied to the skin (Dong Wook Kim, Kun Ho Son, Hyeun Wook Chang, KiHwan Bae and Sam Sik Kang, Arch Pharm Res., 2003, 3, pp 232-236).

6-week-old male ICR mice weighing between 25 g and 28 g (Orient Bio Inc., Korea) were separated for each experimental group. Lactic acid bacteria of Lactobacillus plantarum KC3 (KC3) of Comparative Examples and Examples were diluted in distilled water, and were orally administered at a dose of 300 μl per day for 5 days. In the same way, the administration was performed for a negative administration control group (distilled water administration group) and a positive control material, i.e., cyclooxygenase (COX) inhibitor Indomethacin (10 mg/kg, Sigma).

About 1 hour after administration, 2.5% croton oil (C0421 available from TCI Company, 25 mL) dissolved in acetone was applied evenly to the inside and outside of the right ear of each mice to induce ear edema. After about 4 hours of the induction of edema, the mice were anesthetized with CO₂ and sacrificed. Then, the degree of edema in the ears of the mice was measured according to variations in speed by measuring the thickness of both ears using a thickness gauge (Digimatic thickness gauge, 547-301, Mitutoyo, Japan). Here, based on the degree of and the inhibition rate (%) was calculated based on the degree of edema of the mice in the negative control group (0.5% sodium carboxymethyl cellulose (CMC), Sigma-Aldrich 419273), inhibition rates (%) were calculated, and results thereof are shown in Table 8 below.

3-2 Experiment Results

As a result of the experiment above, it was confirmed that the probiotic lactic acid bacteria from the Lactobacillus plantarum KC3 (KC3) prepared in the Examples of the present disclosure had a significant anti-edema effect in an animal model, i.e., the mice having croton oil-induced inflammation of the ear edema.

TABLE 8
Anti-inflammatory inhibition effect on ear edema

| | | | Inhibition rate |
|---|---|---|---|
| | Dosage for oral | | of ear edema |
| | administration | Thickness | (% of control) |
| Normal control | Distilled water | 0.329 ± 0.032 | — |
| group | | | |
| Control group | Induction of ear edema | 0.806 ± 0.068 | — |
| Positive control | 10 mg/kg BW | 0.684 ± 0.054 | 25.6 ± 2.2 |
| group | | | |
| (Indomethacin) | | | |
| Example 1 | 1 × 10⁹ CFU/mouse | 0.751 ± 0.057 | 11.5 ± 1.0 |
| | 2 × 10⁹ CFU/mouse | 0.693 ± 0.051 | 23.7 ± 2.4 |

### Experimental Example 4: Protective Effect Against Respiratory Damage Caused by Air Pollutants Such as Fine Dust

To confirm protective effects of the probiotic lactic acid bacteria (Lactobacillus plantarum KC3, KC3) against respiratory damage caused by air pollutants such as fine dust, an experiment was carried out as follows by applying the method described in the existing document.

4-1. Experimental Method in Mouse Model Having Respiratory Damage

BALb/c male mice (Orient Bio Inc., 8-week-old) were divided into groups of 6 mice, and for all groups except for the normal group, components of air pollutants, i.e., 10 mg/ml of coal combustible materials, 10 mg/ml of fly ash, and 5 mg/ml of diesel exhaust particle (DEP) were diluted so that a final concentration of Alum was 1%, and so that a final concentration of each component was 1.5 mg/ml for coal combustible material/fly ash and 5 mg/ml for DEP in a mixture. Then, on each of the 4^{th}, 7^{th}, and 10^{th} days from the start of the experiment, 50 μl of the mixture was directly injected into the airway and nose of the experimental animals according to the Intra-Nazal-Trachea (INT) injection method described in the document (Lim et al., Free Radic Biol Med. 25(6), 635-644. (1998), Shin et al., Korean J. Medicinal Crop Sci 27(3), 218-231. (2019)).

For a positive control group (dexamethasone, Sigma D2915), lactic acid bacteria (Lactobacillus plantarum KC3, KC3) was diluted at a concentration of 1×10⁹ CFU/mouse in a solution containing 0.5% sodium carboxymethyl cellulose (CMC, 419273, Sigma-Aldrich), and then orally administered at a dosage of 3 mg per kg of body weight (BW) per day (for 11 days). On the 12^{th} day after the start of the experiment, an autopsy was performed on the mice, and bronchoalveolar lavage (BAL) fluid was collected.

4-2. Measurement of Total Number of Cells in BAL Fluid

4-2-1. Experiment Method

To confirm effects of the samples in Examples above on the total number of cells in BAL fluid, an experiment was carried out as follows by applying the method described in the document (Schins et al., Toxicol Appl Pharmacol. 195(1), 1-11 (2004) and Smith et al., Toxicol Sci, 93(2), 390-399 (2006)).

4-2-2. Experiment Results

The results of measuring the effects of the samples on the total number of cells in the BAL fluid are shown in Table 9 below. Compared to the bronchial damage-induced group by air pollutants, the total number of cells in the BAL fluid was significantly reduced by administration of the probiotic lactic acid bacteria from the Lactobacillus plantarum KC3 (KC3), thereby confirming that the sample had inhibitory activity on the bronchial inflammation.

TABLE 9
Experiment results of effects on the total number of
cells in total bronchoalveolar lavage (BAL) fluid

| | | Inhibition rate |
|---|---|---|
| | Total BAL cell | (based on |
| Division | (×10⁴ cells/ml) | induction group) |
| Normal control group | 16.7 ± 0.93 | |
| Induction group | 81.2 ± 19.8 | |
| Positive control group | 72.6 ± 9.5 | 11% |
| Lactobacillus plantarum | 63.6 ± 8.9 | 22% |
| KC3 | | |

4-3. Measurement of Ratio of Number of Neutrophils to Total Number of Cells in BAL Fluid

4-3-1. Experiment Method

To confirm effects of the samples in Examples above on a ratio of the number of neutrophils to the total number of cells in BAL fluid, an experiment was carried out as follows by applying the method described in the document (Schins et al., Toxicol Appl Pharmacol. 195(1), 1-11 (2004) and Smith et al., Toxicol Sci, 93(2), 390-399 (2006)).

The experiment was carried out in the same manner as in Experimental Examples 4-1 and 4-2. From the collected BAL fluid, the neutrophils were observed after staining according to a Diff-Qick staining method (Takano et al., Am J Respir Crit Care Med, 156(1), 36-42(1997), Hemacolor Rapid staining of blood smear, 1.11661.0001, Merck).

4-3-2. Experiment Results

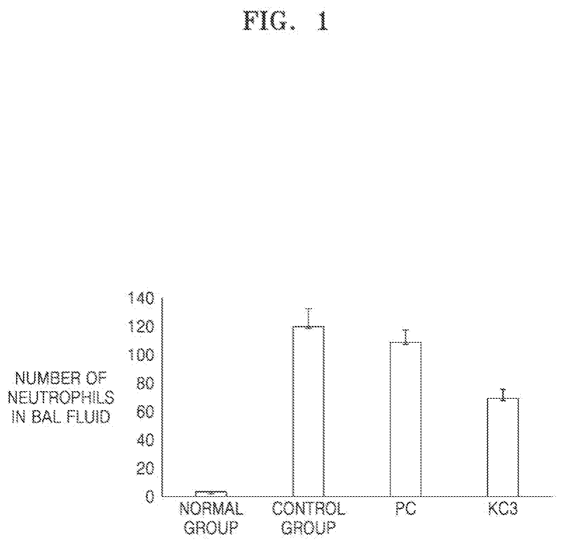

The results of measuring the effects of the samples on the ratio of the number of neutrophils, which are inflammatory immune cells, to the total number of cells in the BAL fluid are shown in Table 10 below. The total number of neutrophils that have increased by air pollutants was significantly reduced by administration of the probiotic lactic acid bacteria from Lactobacillus plantarum KC3 (KC3), thereby confirming that the sample had inhibitory activity on the bronchial inflammation (see Table 10 and FIG. 1).

TABLE 10
Experiment results of effects on number of neutrophils
to total number of cells in BAL fluid

| | Neutrophile No of | Inhibition rate (based on |
|---|---|---|
| Division | BAL cel (400X) | induced group) |
| Normal control group | 0.1 ± 0.1 | |
| Induction group | 119 ± 13 | |
| Positive control group | 108 ± 9 | 10% |
| Lactobacillus plantarum | 68 ± 7 | 43% |
| KC3 | | |

4-4. Measurement of Expression Levels of Inflammation Factors in BAL Fluid

4-4-1. Experiment Method

To confirm effects of the samples in Examples above on expression levels of inflammation factors in the BAL fluid, an experiment was carried out as follows by applying the method described in the document (Brandt E B et al., J. Allergy Clin. Immunol., 132(5):1194-1204, (2013)).

An evaluation test was performed using ELISA to measure the expression levels of inflammation factors, such as IL-17A, TNF-α MIP2, and CXCL-1, in the BAL fluid.

The experiment was carried out in the same manner as in Experimental Examples 4-2 and 4-3, except that the number of cells in the BAL fluid was measured.

The expression levels of IL-17A, TNF-α MIP2, and CXCL-1 in the BAL fluid were measured using ELISA. IL-17A antibodies (M1700, R&D Systems, Minneapolis, USA), TNF-α antibodies (MTA00B, R&D Systems, Minneapolis, USA), MIP2 antibodies (MM200, R&D Systems, Minneapolis, USA), and CXCL-1 antibodies (MKC00B, R&D Systems, Minneapolis, USA) were diluted with a buffer solution and coated micro-wells, and then cultured at 4° C. for 16 hours. Each well was washed with a washing buffer solution three times, and 10-fold diluted serum was dispensed at 100 μl per well.

After being left at room temperature for one hour, the wells were washed twice. Then, 100 μl of Avidin-HRP-conjugated antibodies (DY007, R&D Systems, Minneapolis, USA) was treated thereon, and the wells were left again at room temperature for one hour, followed by washing again. A TMB substrate solution (DY007, R&D Systems, Minneapolis, USA) was dispensed at 100 μl per well, and the well plate was left in shadow for 30 minutes. Then, 50 μl of a stopping solution (DY007, R&D Systems, Minneapolis, USA) was treated thereon, and absorbance of the cells was measured at 450 nm.

4-4-2. Experiment Results

The results of measuring the levels of inflammation biomarkers, such as IL-17A, TNF-α, MIP2, and CXCL-1, in the BAL fluid are shown in Table 11 below. The levels of the inflammation biomarkers (i.e., IL-17A, TNF-α, MIP2, and CXCL-1) that have increased by air pollutants were significantly reduced by the administration of the probiotic lactic acid bacteria from Lactobacillus plantarum KC3 (KC3), thereby confirming that the sample significantly defended against bronchial inflammation and damage.

TABLE 11
Experiment results of effects on expression
of inflammation factors in BAL fluid

| | Concentration (pg/ml)/inhibition | | | |
|---|---|---|---|---|
| | rate (%) based on induced group | | | |
| Division | IL-17A | TNF-α | MIP2 | CXCL-1 |
| Normal control | 6.3 ± 0.9 | 79 ± 6 | 49 ± 4 | 87 ± 4 |
| group | | | | |
| Induction group | 13.4 ± 1.5 | 366 ± 45 | 68 ± 7 | 327 ± 41 |
| Positive control | 7.0 ± 0.8 | 121 ± 14 | 55 ± 9 | 174 ± 15 |
| group | (47%) | (66%) | (19%) | (46%) |
| Lactobacillus | 6.9 ± 0.7 | 134 ± 13 | 59 ± 7 | 121 ± 16 |
| plantarum | (48%) | (63%) | (13%) | (63%) |
| KC3 | | | | |

### Mode of Disclosure

Hereinafter, a formulation method and kinds of carriers will be described, but the present disclosure is not limited thereto. The representative preparation examples will be described.

Preparation examples of a composition including a sample of the present disclosure are described, but the present disclosure is not limited thereto. These preparation examples are merely described in detail.

### Formulation Example 1. Preparation of Powder

| | |
|---|---|
| New lactic acid bacteria from KC3 | 20 mg |
| Lactose | 100 mg |
| Talc | 10 mg |

The components above were mixed and filled in an airtight bag to prepare a powder.

### Formulation Example 2. Preparation of Tablet

| | |
|---|---|
| New lactic acid bacteria from KC3 | 10 mg |
| Corn starch | 100 mg |
| Lactose | 100 mg |
| Magnesium stearate | 2 mg |

The tablet was prepared by mixing the above components and en-tableting the same, according to an existing tablet formation method.

### Formulation Example 3. Preparation of Capsule

| | | |
|---|---|---|
| New lactic acid bacteria from KC3 | 10 | mg |
| Crystalline cellulose | 3 | mg |
| Lactose | 14.8 | mg |
| Magnesium stearate | 2 | mg |

A capsule was prepared by mixing the components above and filling a gelatin capsule with the mixture, according to an existing capsule formation method.

### Formulation Example 4. Preparation of Injection

| | | |
|---|---|---|
| New lactic acid bacteria from KC3 | 10 | mg |
| Mannitol | 180 | mg |
| Sterile distilled water for injection | 2,974 | mg |
| Na₂HPO₄, 12H2O | 26 | mg |

According to an existing injection formation method, an injection was prepared based on the component contents above per 1 ampoule (2 ml).

### Formulation Example 5. Preparation of Liquid Formulation

| | | |
|---|---|---|
| New lactic acid bacteria from KC3 | 20 | mg |
| Isomerized glucose syrup | 10 | g |
| Mannitol | 5 | g |

| | |
|---|---|
| Purified water | optimum amount |

According to an existing preparation method for a liquid formulation, each component was added to purified water and dissolved therein, and an optimum amount of lemon flavor was added and mixed with the components above. Then, purified water was added thereto so that the total volume was adjusted to 100 ml, and the resultant solution filled in a brown bottle and sterilized, thereby preparing a liquid formulation.

### Formulation Example 6. Preparation of Health Food

| New lactic acid bacteria from KC3 | 1,000 | mg |
|---|---|---|
| Vitamin mixture | optimum amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| nicotinic acid amide | 1.7 | mg |
| Folic acid | 50 | μg |
| calcium pantothenate | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

A compositional ratio of vitamins and minerals in the mixture was provided as a preferable example using components relatively suitable for health food. However, such a mixture ratio may be arbitrarily modified considered without departing from the spirit and scope of the present disclosure.

### Formation Example 7. Preparation of Health Beverage

New lactic acid bacteria from KC3 - - - 1,000 mg

Citric acid - - - 1,000 mg

Oligosaccharide - - - 100 g

Plum concentrate - - - 2 g

Taurine - - - 1 g

By addition of purified water - - - 900 ml in total

According to an existing method of preparing a health beverage, the components above were mixed and heated at 85° C. for about one hour while stirring. The prepared solution was filtered and collected in a sterilized 2 L container. The container was sealed, sterilized, and stored in a refrigerator to be used for the preparation of a health beverage composition.

The compositional ratio above was provided as a preferable example using components relatively suitable for beverages. However, depending on regional and ethnic preferences in terms of a demand class, a country of demand, and a purpose of use, such a mixing ratio may be arbitrarily modified.

As described above, the present disclosure may be varied in many ways, and such variations are not construed as a departure from the spirit and scope of the present disclosure. It would be apparent for those skilled in the art to understand that all such modifications intended to be included within the scope of the following claims of the present disclosure.

### INDUSTRIAL APPLICABILITY

As described above, it was confirmed that the composition is useful as a pharmaceutical composition or a health functional food for ameliorating an immune disorder and preventing and treating a respiratory inflammatory disease by confirming that the lactic acid bacteria from the Lactobacillus plantarum KC3 strain exhibit excellent activities of ameliorating an immune disorder and inhibiting a respiratory inflammatory disease through animal experiments, such as an experiment on the characteristics of the new lactic acid bacteria from the Lactobacillus plantarum KC3 (Experimental Example 1); an experiment on the inhibitory effects of probiotics on the expression of inflammatory cytokines in the intestines (Experimental Example 2); an experiment on the anti-inflammatory effect on ear edema (Experimental Example 3); and an experiment on the defense effects against respiratory damage caused by air pollutants such as fine dust (Experimental Example 4). Accordingly, the composition may be useful as a pharmaceutical composition, a health functional food, or a health supplement food for ameliorating immune disorders and preventing and treating respiratory inflammatory diseases.

1. A pharmaceutical composition for ameliorating immune disorders and preventing or treating respiratory inflammatory diseases, the pharmaceutical composition comprising, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

2. The pharmaceutical composition of claim 1, wherein
the immune disorders are selected from diseases caused by a decrease in immune function due to anticancer therapy such as chemotherapy and radiation therapy, diseases caused by decreased immunity after bone marrow transplantation, AIDS due to damage to an immune system, and cancer diseases caused by a decrease in immune function.

3. The pharmaceutical composition of claim 1, wherein
the respiratory inflammatory diseases are selected from the group consisting of rhinitis, otitis media, sore throat, tonsillitis, pneumonia, asthma, and chronic obstructive pulmonary disease (COPD).

4. A health functional food for ameliorating immune disorders and preventing or treating respiratory inflammatory diseases, the health functional food comprising, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

5. The health functional food of claim 4, wherein
the health functional food is in the form of powder, granules, tablets, capsules, pills, suspensions, emulsions, syrups, tea bags, leached teas, or health beverages.

6. A health supplement food for ameliorating immune disorders and preventing or treating respiratory inflammatory diseases, the health supplement food comprising, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

7. A food for ameliorating immune disorders and preventing or treating respiratory inflammatory diseases, the food comprising, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

8. A food additive for ameliorating immune disorders and preventing or treating respiratory inflammatory diseases, the food additive comprising, as an active ingredient, one or more selected from the group consisting of a Lactobacillus plantarum KC3 strain (also referred to as “CKDB-KC3”, Accession number: KCTC13375BP), a culture thereof, a concentrate of the culture, and a dried material of the culture.

9. A method of ameliorating immune disorders and treating a patient having a respiratory inflammatory disease, the method comprising administering one or more selected from the group consisting of a Lactobacillus plantarum KC3 strain (also referred to as “CKDB-KC3”, Accession number: KCTC13375BP), a culture thereof, a concentrate of the culture, and a dried material of the culture, to a patient having an immune disorder and a respiratory inflammatory disease.

10. Use of one or more selected from the group consisting of a Lactobacillus plantarum KC3 strain (also referred to as “CKDB-KC3”, Accession number: KCTC13375BP), a culture thereof, a concentrate of the culture, and a dried material of the culture, for preparation of a drug for amelioration of immune disorders and treatment of respiratory inflammatory diseases.


SEQUENCE LISTING (7 sequences)

SEQ ID NO: 1 - DNA, 1509 bp, Artificial
  16s rRNA of L. plantarum KC3 (KCTC13375BP)
tatggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaca

SEQ ID NO: 2 - DNA, 20 bp, Artificial
  beta-actin forward primer sequence
aggtgaaggtcggagtcaac

SEQ ID NO: 3 - DNA, 20 bp, Artificial
  beta-actin reverse primer sequence
gctcctggaagatggtgatg

SEQ ID NO: 4 - DNA, 20 bp, Artificial
  interleukin 1 beta forward primer sequence
acagatgaagtgctccttcc

SEQ ID NO: 5 - DNA, 20 bp, Artificial
  interleukin 1 beta reverse primer sequence
gtcggagattcgtagctgga

SEQ ID NO: 6 - DNA, 20 bp, Artificial
  tumor necrosis factor alpha forward primer sequence
gaaagcatgatccgggacgt

SEQ ID NO: 7 - DNA, 20 bp, Artificial
  tumor necrosis factor alpha reverse primer sequence
gatggcagagaggaggttga